(19) United States
(12) Patent Application Publication
(10) Pub. No.: US 2019/0262500 A1
(43) Pub. Date: Aug. 29, 2019

(54) ALTERNATING BLOCK POLYURETHANES AND THE USE IN NERVE GUIDANCE CONDUITS

(71) Applicants: Kaitian Xu, State College, PA (US); Zhiyuan Lan, Lowell, MA (US)

(72) Inventors: Kaitian Xu, State College, PA (US); Zhiyuan Lan, Lowell, MA (US)

(21) Appl. No.: 15/343,206

(22) Filed: Nov. 4, 2016

(51) Int. Cl. A61L 27/18; A61L 27/54; A61L 27/58; C08G 18/40; C08G 81/00; A61B 17/11

(52) U.S. Cl. CPC A61L 27/18; A61L 27/54; A61L 27/58; C08G 2230/00; C08G 81/00; A61B 17/1128; A61L 2430/32; C08G 18/4018

(57) ABSTRACT

This invention of new biomaterials of alternating block polyurethanes (AltPU) based on biodegradable polyester blocks and hydrophilic blocks such as polyethers are created through a selectively coupling reaction between aliphatic polyester diols and diisocyanate-terminated hydrophilic polyethers or between aliphatic polyester diols and diisocyanate-terminated aliphatic polyester blocks under catalysis of organic tin compounds. AltPU possess well-controlled and defined chemical structures as well as regular polymer chain architecture and surface microstructures. The alternating block polyurethane designs endow materials with more special and intriguing properties, such as better biocompatibility, higher hydrophilicity, and favorable mechanical and material processing properties. Medical devices made of AltPU biomaterials show outstanding performance in peripheral nerve repair. In peripheral nerve repair (NGC), NGCs made of AltPU exhibit even better repair results than autograft, without adding any additional growth factors or proteins on SD rat model. The NGCs can also contain bioactive substances. The AltPU biomaterials can be widely used for many medical and non-medical applications including but not limited to tissue regeneration of soft and hard tissues, medical tubings and catheters, device coatings, and other applications.

### STATEMENT REGARDING FEDERALLY SPONSORED

This invention was not made with federally government sponsored research.

### FIELD OF THE DISCLOSURE

The present disclosure relates to alternating block polyurethanes (abbreviation: AltPU), their application as nerve repair conduit. More particularly, the present disclosure relates to the design of biodegradable block polyurethanes with alternating arrangement of the block segments such as PCL, PLA, PHA and PEG but also including random arrangement of the block segments. Some such polyurethanes were obtained by a selectively coupling reaction between aliphatic polyester diols, aliphatic diols, and PEG diisocyanates and other aliphatic diisocyanates. The thus obtained materials were used as peripheral nerve regeneration materials for fabrication of peripheral nerve guidance conduit (NGC), and the NGC fabrication methods.

### BACKGROUND

Biodegradable block polyurethanes are a class of biomaterials which are widely used in tissue engineering, regenerative medicine, controlled drug delivery, wound healing, and other applications, due to their excellent hemocompatibility, mechanical and processing properties [1]. However, almost all of the traditional block polyurethanes were synthesized via the coupling reaction of terminal hydroxyl group of aliphatic polyester diols with or without PEG by using diisocyanates as coupling agents. Even though this method would provide the materials with improved properties, this approach actually lacks the block selectivity and provides the copolymers with blocks connected in a random manner (i.e. traditional or random block polyurethanes, abbreviation: RanPU) (FIG. 1). The random block structure results in a difficulty of fine-tuning the material properties.

In this invention, alternating block polyurethanes (AltPU) are developed as a new class of block polyurethanes comprised of an alternating arrangement of the blocks (FIG. 2), such as polycaprolactone (PCL), poly(lactic acid) (PLA), polyhydroxyalkanoates (PHA) and poly(ethylene glycol) (PEG) blocks. These block urethanes possess a pre-determined chemical structure as well as a regular physical microstructure [2-5]. The alternating structure is created via selectively coupling reaction between aliphatic polyester diols and PEG or aliphatic diisocyanate. Additionally, not intending to be bound by theory, it is believed that the alternating structure confers the materials with many intriguing properties.

Also described are some representative biomedical applications of AltPU, especially for the fabrication of peripheral nerve guidance conduits (abbreviated as NGC; or nerve repair conduit) for peripheral nerve repair [6-7].

Peripheral nerve defect is a very common clinical trauma and often leads to permanent disability of feeling and movement function in affected patients, which affects approximately 360,000 people every year in the United States. Transplantation of autologous nerve graft (autograft) has typically been used for the repair of injured peripheral nerves as a first line therapy. However, there are many disadvantages with this method, including size mismatch between the defect nerve and graft nerve, a second surgical step for the extraction of donor nerves, a shortage in the supply of donor grafts, donor site morbidity, inadequate return of function and aberrant regeneration. Due to host immunogenic rejection of the donor graft, the method of using allografts achieves very few successes in clinical practice. Morbidity of harvesting donor grafts hinders development of the muscle and vein grafts during repair of severed peripheral nerves. Furthermore, none of these surgical autologous approaches has resulted in axonal connections. To overcome these problems, an alternative approach would be to use a synthetic biodegradable nerve repair scaffold serving to both promote nerve regeneration and provide a pathway for nerve outgrowth.

In the 1990's, Schakenraad and Robinson [8, 9] performed systematic research on nerve regeneration using scaffolds based on biodegradable copolymers of DL-lactide and caprolactone. Based on this work, the first commercialized artificial nerve repair scaffold was prepared from the biodegradable copolymers of DL-lactide and caprolactone [P(DLLA-co-CL)], which are now used clinically under the trade name Neurolac®.

In current state-of-the-art treatment for nerve trauma, a few biodegradable nerve guidance conduits (NGC) are commercially available for clinical use [10], i.e. Neurolac@ (Polyganics), NeuraGen (Integra LifeSciences), NeuraWrap (Integra Life Sciences), NeuroMend (Collagen Matrix), GEM™ Neurotube (Synovis Micro), Avance@Nerve Graft (AxoGen), NeuroFlex (Collagen Matrix), and Salutunnel (Salumedica). All of them are made from synthetic biodegradable polymers such as poly(L-lactic acid) (PLLA), poly (D, L-lactic-co-glycolic acid) (PLGA), polycaprolactone (PCL), polyvinyl alcohol (PVA) or naturally originated polymer such as collagen. Although these nerve conduits provide an alternative surgical option over autografts, their performance still remains inferior to autografts in the functional recovery of injured nerves, even over short injury gaps. Overall, the currently available nerve guidance conduit products have a suboptimal regenerative capacity and poor functional recovery compared to autograft in peripheral nerve repair treatment.

Biodegradable polyurethanes (PU), however, have been recently explored as novel biomaterials due to their excellent mechanical and processing properties and good biocompatibility. Even though much effort has been spent in applying polyurethanes for different biomedical purposes, there is a scarcity of research on nerve regeneration and synthetic nerve repair conduits based on polyurethanes as the scaffold materials. The first attempt using polyurethanes in preparation of a double-layered nerve conduit appeared in 1990 by Pennings [11], in which a mixture of biodegradable polyurethane and poly(L-lactide) served as the outer microporous layer of the double-layered conduit. Although this dual-component polyurethane based nerve conduit demonstrated high performance in nerve regeneration across an 8-mm gap, the conduit failed to degrade completely and was marred by cytotoxic degradation products. This led to the emergence of another nerve guide conduit composed of semi-crystalline poly-L-lactide and polycaprolactone (50/50), which showed much improved nerve regeneration through the conduit [12]. However, remnants of the biomaterial lingered around the regenerating nerve up to 2 years post implantation. A comprehensive review on nerve repair using biodegradable artificial nerve guidance conduits was addressed by Johnson in 2008 [13]. Furthermore, a systematic review on animal models used to study nerve regeneration was reported by Windebank [14].

Wang et al [15, 16] also prepared double layered nerve conduit with collagen inner layer and a PCL and PEG based traditional biodegradable polyurethane outer layer for nerve repair. No satisfactory results were achieved in SD rat animal test.

Yang et al [17] described an investigation on the application of a crosslinked urethane-doped biodegradable polyester (CUPE) scaffolds for nerve regeneration. The CUPE nerve guides were also evaluated in vivo for the repair of a 1 cm rat sciatic nerve defect. Histological evaluations revealed a collapse of the inner structure of the CUPE nerve guides, fiber populations and densities analysis gave fairly good results after 8 weeks of implantation.

Utilizing the disclosed alternating block polyurethanes (AltPU) contained herein, we have explored AltPU in different biomedical applications, especially nerve repair [6, 7, 18]. PCL and PEG based alternating block polyurethanes (PUCL-alt-PEG) (FIG. 2) possess obvious better hemocompatibility than the random block counterpart (FIG. 3) and much better hemocompatibility than biodegradable polymers such as poly(L-lactic acid) (PLLA), poly(D, L-lactic-co-glycolic acid) (PLGA), polycaprolactone (PCL), or collagen implant materials. Our results also suggest that AltPU possesses better cytocompatibility with neural rat glial cells than the traditional random block counterpart PUCL-ran-PEG and PCL.

During degradation of PLLA, PLGA, and/or PCL-based implant materials, significant pH value changes (increased acidity) can cause inflammation in local tissue and negatively affect the medical performance. However, nerve repair conduit made of the alternating block polyurethanes (AltPU) displayed only a very mild pH value change during degradation which is even less than their random block polyurethane counterparts, PUCL-ran-PEG and PCL. The small changes in the pH values caused by the degradation of polyurethane nerve guidance conduit may be due to the urethane chemical structure, which simultaneously generates acidic carboxylic groups and basic amine groups during degradation This is unlike the aliphatic polyesters such as PCL and PLA, that generate only acidic carboxylic groups during degradation, resulting in significant reduction of pH in the area local to the aliphatic polyester implants. Not intending to be bound by theory, it is believed that the small change in pH value of the PU scaffolds contributes to the reduction of the inflammatory risk when compared to implants made from PLLA, PLGA, PCL-based materials.

Natural polymers such as collagen type I and decellularized small intestinal submucosa have been used to construct NGCs such as NeuroMatrix®, NeuroFlex®, and NeuroGen®. However, these natural polymers suffer from the following concerns: 1) undesirable immune response and requirement of long-term administration of immunosuppressant; 2) high cost; 3) variable physiochemical properties and degradation properties; and 4) risk of infection and disease transmission.

Previous synthetic NGCs are usually made of biodegradable polymers such as poly(L-lactic acid) (PLLA), poly (D, L-lactic-co-glycolic acid) (PLGA), polycaprolactone (PCL), polyvinyl alcohol (PVA) and poly(lactide-co-caprolactone) (PLCL). Synthetic polymers are advantageous in term of alleviating the concern on batch-to-batch variations and usually offer excellent processability and provide excellent control on material mechanical, degradable, and biological properties. However, the common problems are 1) either too fast degradation, which causes early collapse (few weeks) or too slow degradation (>8 months, even >1 year) that is concomitant with incomplete or fragmental degradation; 3) acidic degradation products; 4) high rigidity of the NGC that may result in nerve stumps being torn out of the NGC lumen during regeneration; 5) poor kink resistance that causes lumen occlusion and prevent nerve regeneration; and 6) severe inflammatory responses that cause fibrosis and present nerve regeneration.

Therefore, developing ideal synthetic nerve guidance conduits that can address all the above concerns for peripheral nerve regeneration is of great medical and economical significance, especially in the regeneration of critically sized nerve injury.

### SUMMARY

The present disclosure describes a series of alternating block polyurethanes (AltPU) with alternating arrangement of the blocks (FIG. 2), such as polycaprolactone (PCL), polylactide (PLA), and poly(ethylene glycol) (PEG) blocks, which possess well-defined chemical structures as well as intriguing microstructures and surface topologies. The alternating structure could be provided via a selective coupling reaction between aliphatic polyester diols and PEG diisocyanate by using suitable catalysts. The obtained materials, when fabricated for biomaterial applications, have improved hemocompatibility, biocompatibility, surface properties, mechanical properties, processing ability, biodegradation properties, minimal pH change due to degradation by-products, etc.

This disclosure also describes fabricated forms of the AltPU, specifically for biomedical applications, as well as other applications requiring the physical and material properties of AltPU.

Nerve guidance conduits fabricated from the alternating block polyurethane PUCL-alt-PEG showed satisfactory nerve regeneration associated with excellent neurogenesis, and functional rehabilitation of neurons after 32 weeks post implantation. The amphiphilic PUCL-alt-PEG scaffolds exhibited comparable or slightly better nerve regeneration than autografts, which are widely used clinically. Further, such PUCL-alt-PEG nerve repair scaffolds can be used in large nerve gap repair.

### BRIEF DESCRIPTION OF THE DRAWINGS

FIG. 1. Synthetic strategy and structure comparison of random block polyurethane (RanPU) (upper portion) and alternating block polyurethane (AltPU) (lower portion).

FIG. 2. Synthetic strategy and structure of alternating block polyurethanes (AltPU).

FIG. 3. Synthetic strategy and structure of random block polyurethanes (RanPU).

FIG. 4. Schematic illustration of nerve repair microsurgery (a). SEM images of polyurethane nerve guidance scaffold with controlled microporosity: (b) cross-sectional morphology; (c) wall microstructure. This figure is consistent with the embodiment of Example 2.

FIG. 5. Walking track analysis, (a) sciatic function index (SFI) values of rats at 2, 4, 8, 10, 14 weeks after implantation; (b) foot prints at 4 (b1), 8 (b2), 14 (b3) week postoperatively. This figure is consistent with the embodiment of Example 2.

FIG. 6. The CMAP (Compound Muscle Action Potentials) signals were compared with the animal's contralateral control and expressed as the CMAPs ratio. This figure is consistent with the embodiment of Example 2.

FIG. 7. NGC degradation and regenerated nerves of PUCL-alt-PEG scaffolds: (a) implanted in rat at 9 week postoperatively (surrounded with abundant capillaries); (b) a regenerated nerve after taking off the scaffolds; (c) scaffolds degraded completely at 32 week implantation with a mature regenerated nerve. This figure is consistent with the embodiment of Example 2.

FIG. 8. Cross-section of the mid-section of regenerated nerve at 9 week postoperatively: (A, F) PUCL-alt-PEG scaffold; (B, G) PUCL-ran-PEG scaffolds; (C, H) autograft; (D, I) PCL scaffold; (E, J) silicone tube. (A, E) HE staining; (F, □J) anti-neurofilament staining. This figure is consistent with the embodiment of Example 2.

FIG. 9. Ammonia silver staining of the longitudinal section of mid-section of nerves at 14 weeks post implantation: (A) PUCL-alt-PEG scaffold; (B) PUCL-ran-PEG; (C) autograft; (D) PCL; (E) silicone. This figure is consistent with the embodiment of Example 4.

FIG. 10. Masson's trichrome staining of gastrocnemius muscle cross-section at 14-week implantation. (A) PUCL-alt-PEG scaffold; (B) PUCL-ran-PEG scaffold; (C) autograft; (D) PCL; (E) silicone tube; (F) negative control groups, n=4; *p<0.05; scale bar=60 μm. This figure is consistent with the embodiment of Example 2.

FIG. 11. Surface morphology of polyurethane scaffolds degradation at 9th week implantation in vivo, upper row: (A) a PUCL-alt-PEG scaffold at low magnification; (B) higher magnification 2000 of dotted box from panel A star; (C) HE staining 100 from dotted box from panel A; lower row: (D) PUCL-ran-PEG scaffold at low magnification; (E) higher magnification 2000 of dotted box from panel D star; (F) HE staining 100 from dotted box from panel D. Black arrow, blood vessels; white arrow, connective tissues. This figure is consistent with the embodiment of Example 2.

### DETAILED DESCRIPTION OF THE DISCLOSURE

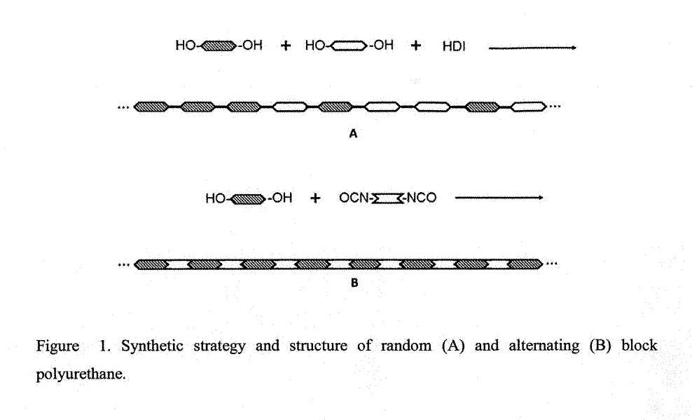

The present disclosure provides preparation methods and biomedical applications of alternating block polyurethanes (AltPU). AltPU are a class of block polyurethanes with alternating arrangement of the blocks, such as PCL, PLA and PEG blocks, which possess determined and regular macromolecular structure and architecture. However, traditional block polyurethanes consist of blocks connected at a random manner, i.e. random block polyurethanes (RanPU). Their macromolecular structure and architecture are important factors that determine the material and physical properties and biological performance. The architecture of random block polyurethanes and alternating block polyurethanes are compared in FIG. 1.

The alternating block polyurethanes architectures are created through a selectively coupling reaction between aliphatic polyester diols and diisocyanate-terminated hydrophilic segments such as PEG, or between aliphatic polyester diols and diisocyanate-terminated aliphatic polyester segments. This chemical reaction can result in only alternating connection of the blocks, thus creating alternating block polyurethanes with a well-controlled regular structure (FIG. 1). However, traditional random block polyurethanes are prepared via the coupling reaction of terminal hydroxyl group of aliphatic polyester diols with or without hydrophilic blocks such as PEG by using diisocyanates as coupling agents. This approach lacks the block selectivity and provides the polyurethanes with blocks connected at a random manner (FIG. 1). Thus, structure of random block polyurethanes can be controlled only in a rough way that is not able to construct the architecture in an accurate or alternating arrangement. The reaction processes are compared in FIG. 1.

The reactions are carried out either in bulk or in organic solvents and typically need to be performed under inert atmosphere without moisture conditions. Tin catalysts such as tin(II) 2-ethylhexanoate (SnOct2), ditin butyldilaurate are typically necessary for the reaction. Hydroxyl group and isocyanate group should typically be equal molar ratio. The reaction conditions are typically at 30˜100 ° C. for 8˜72 h.

The prepared AltPU possesses a well-controlled and pre-determined chemical structure, macromolecular architecture as well as regular surface microstructure. AltPU shows higher molecular weight and crystallinity, higher surface energy, more regular and stable ravine-like surface patterns in comparison to RanPU. The alternating structures enhance micro-phase separation thus allowing PEG segments mobilizing onto the material surfaces, resulting in the relatively higher surface energy. The higher crystallinity enhances the mechanical strengths and stabilizes the ravine patterns. The alternating structures result in a ravine surface with regular patterns. The roughness of the surface was further investigated by atomic force microscope (AFM). Height images of the AltPU and RanPU with the same chemical composition were recorded. It was found that alternating AltPU presents a Ra (average roughness) of 101.8 nm and a Rmax (maximum roughness) of 762.9 nm, which are much higher than RanPU with a Ra of 47.6 nm and a Rmax of 387.9 nm.

Not intending to be bound by theory, it is believed that the regular structures endow materials with more special and intriguing properties, such as better cytocompatibility and hemocompatibility, mild pH change in degradation, mechanical and shape-forming properties, well-controlled degradation rates, and versatility for a broad range of biomedical and other applications. AltPU cannot only be used in applications that use traditional block polyurethanes and polylactones but also in applications that can benefit from more special and intriguing properties. For example, with the regular surface micropattern that is naturally formed during the reaction, AltPU films and scaffolds possesses much better hemocompatibility and cytocompatibility with such as fibroblasts and neural rat glial cells than traditional block polyurethane counterpart and polylactones.

Nerve guidance conduits (NGC) are made from AltPU are demonstrated. Other medical devices, such as tissue engineering scaffolds for cellular ingrowth, cartilage reconstruction, organ replacement and repair, ligament and tendon repair, bone reconstruction and repair, skin reconstruction and repair, vascular graft, and coronary stents, can also be made from AltPU biomaterials. The mentioned medical devices and scaffolds of alternating block polyurethanes (AltPU) are fabricated using the common methods such as salt leaching, freeze-drying, electrospinning, extrusion, molding, casting and even 3-dimensional (3D) printing or additive manufacturing.

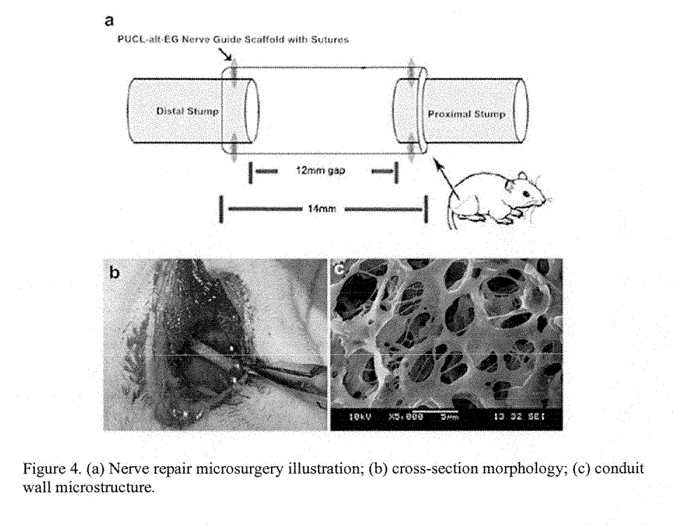

Nerve repair conduits made from alternating block polyurethanes (AltPU) exhibit comparable or even better repair effects than autografts in SD rat model, through a systematic investigation and comparison of nerve repair for AltPU, RanPU, autograft, PCL, silicone tube, and negative control, by analysis of sciatic function index (SFI), histological assessment including HE staining, immunohistochemistry, ammonia silver staining, Masson's trichrome staining, as well as TEM observation (FIGS. 4-10).

### EXAMPLE 1

Example of synthesis of PCL and PEG based alternating block polyurethanes (PUCL-alt-PEG) and random block polyurethanes (PUCL-ran-PEG).

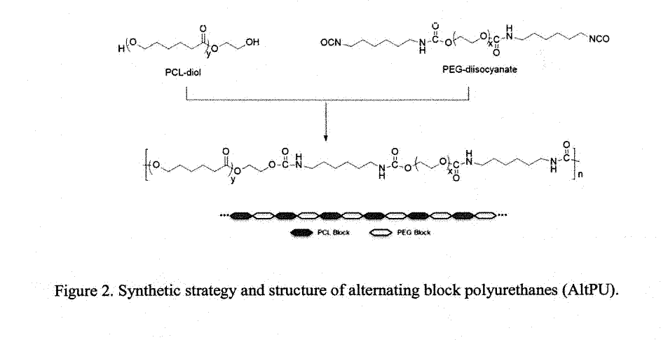

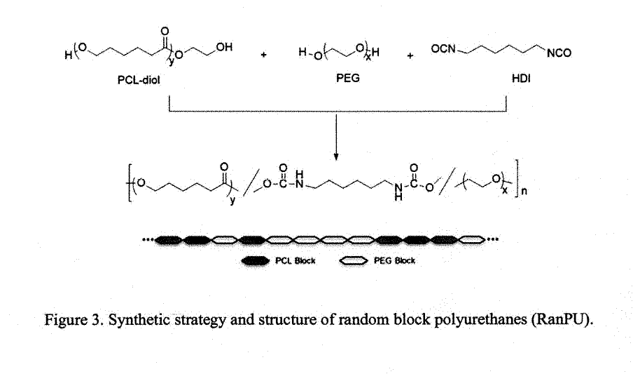

Diisocyanate-terminated PEG was synthesized according to Schouten et. al., Biomaterials 2005, 26, 4219-4228. PCL-diol was first dissolved in 1,2-dichloroethane in a three-neck flask. The prepared PEG-diisocyanate then was dropped slowly into the flask. After 8 h˜72 h reaction at 30° C.˜100° C., the alternating block polyurethane was achieved, where the synthetic reaction is briefly described in FIG. 2. PUCL-ran-PEG was synthesized from PCL-diol and PEG with stannous catalyst using HDI as a coupling reagent. The amount of HDI added was equivalent to the —OH group in the solution. The reaction mixture was stirred at 30° C. to 100° C. under a nitrogen atmosphere for 8 h˜72 h. The product then was collected and dried under vacuum to a constant weight. The synthetic reaction is described in FIG. 3.

### EXAMPLE 2

### Nerve Repair Test

Fabrication of polyurethane nerve guidance conduit

A porous polyurethane nerve guidance conduit was prepared using a dip-coating and salt-leaching method, and a stainless steel wire with an outer diameter of 1.5 mm was used as a mold. The resulting polymer coatings on the mold were then subject to air-drying for 2 days, vacuum-drying for 2 days, followed by salt-leaching in deionized water, freeze-drying, and demolding to obtain a porous nerve guidance conduit.

In SD rat animal models of nerve repair trials, a systematic investigation and comparison of nerve repair is made using scaffolds made from PUCL-alt-PEG and PUCL-ran-PEG (Example 1), autograft, PCL, silicone tube, and negative control. Eighty adult SD rats weighing 200-250 g were used to evaluate the nerve repair. Animals were divided into 5 groups, each with 15 rats. The nerve regeneration capabilities of hydrophilic PUCL-alt-PEG and PUCL-ran-PEG (Example 1) conduits were compared with those of autograft nerve, PCL with similar dimensions (inner diameter, about 1.3 mm; wall thickness about 0.4 mm), non-porous silicone tube (inner diameter 1.5 mm; wall thickness 0.4 mm) and an untreated group (negative control). Defects of 12 mm in sciatic nerves created by surgical removal of the nerve tissue were repaired with the nerve conduits. A schematic illustration of the nerve repair microsurgery, NGC and the porous microstructure is depicted in FIG. 4. Animals were anesthetized with 50 mg/kg body weight pentobarbital sodium. Sciatic nerve on right side was exposed, and a 12 mm segment of nerve was removed from the mid-thigh level. A 14 mm conduit or the removed nerve itself was interposed between the proximal and distal stumps with 8-0 absorbable PLGA at each junction. Following implantation, muscle incision was closed using a 5-0 chromic gut suture and the skin was closed with 2-0 silk suture. Each rat received one implant, which was removed at various time intervals. Postoperatively, each animal was housed in a single cage with free access to food and water. The animals were intensively examined for signs of autotomy and contracture. At each time interval, sciatic function index, electrophysiological and histomorphometric analysis were performed to evaluate the efficiency of nerve repair. All animal experiments were conducted according to the ISO100993-2:1992 animal welfare requirements.

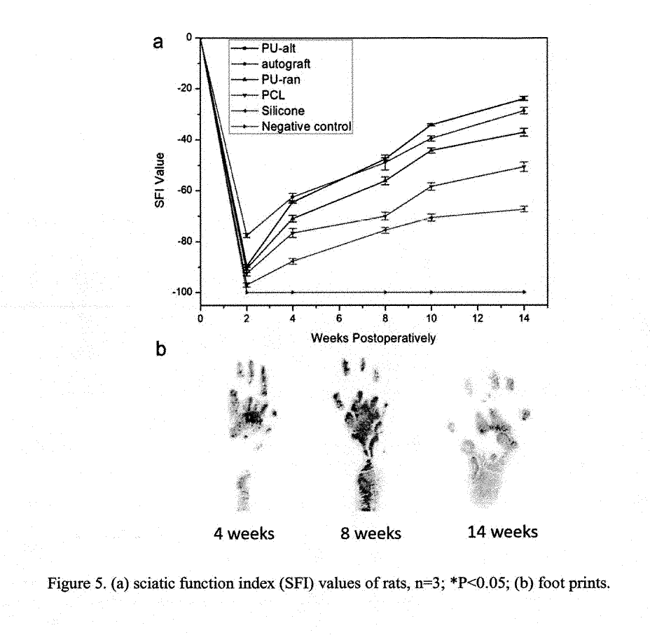

Functional Behavior Training and Electrophysiological Assays for Nerve Repair: The SD rat (sciatic nerve defect) model was used to evaluate the peripheral nerve regeneration capabilities of the six prepared groups, i.e. PUCL-alt-PEG, autograft, PUCL-ran-PEG scaffold, PCL scaffold, silicone tube and negative control. In order to determine the functional characteristics of our scaffolds, PU and PCL nerve guides of 1.28 mm in diameter were determined to be strong enough to maintain an intact structure throughout the surgical implantation process. At predetermined periods (2, 4, 8, 10 and 14 week postoperatively), the nerve regeneration was evaluated by walking track analysis. Sciatic Function Index (SFI) values of different groups are compared in FIG. 5. It is disclosed that a SFI value of −24% recovery was observed in the PUCL-alt-PEG group after 14 weeks post implantation, which was higher than the (−28)% recovery SFI value of the autograft group and much better than the −35% recovery SFI value of PUCL-ran-PEG, and also the SFI values of PCL, silicone tube, and negative control groups. The footprints of animals implanted with PUCL-alt-PEG scaffolds at 4, 8, 14 week postoperatively are also displayed in FIG. 5. It can be seen that at 2 and 8 week, the footprint images were quite narrow and abnormal. The motor function was not at all recovered at this time. At the 14th week mark, the footprint images returned to normal, indicating that the nerve motor function recovered significantly.

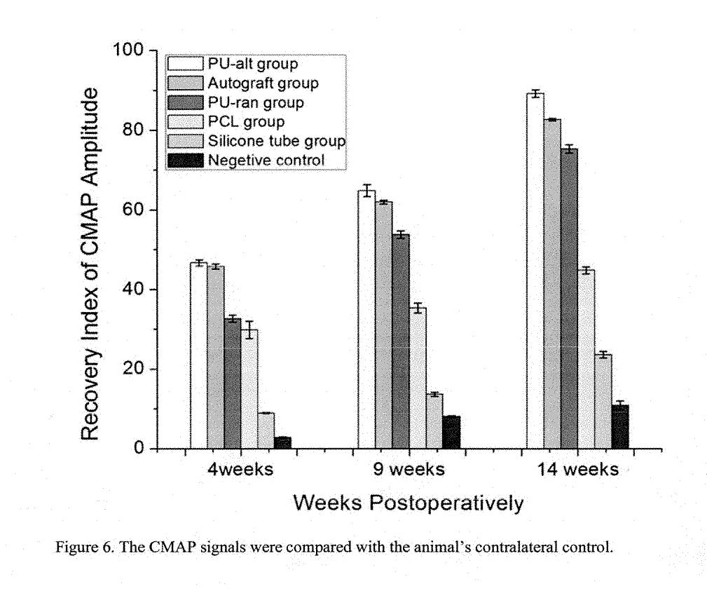

The signals of CMAPs and the corresponding action potentials of PUCL-alt-PEG, PUCL-ran-PEG, autograft, PCL scaffolds, silicone tube and negative control after 4, 8, and 14 weeks implantation were also compared with the signals of the rats' normal sides (FIG. 6). The action potentials were clearly noticeable in the PUCL-alt-PEG, PUCL-ran-PEG, autograft, and PCL scaffold groups after 4 weeks, indicating rapid functional recovery of the injured nerves. The potentials became more intense after 9 and 14 weeks, indicating notable nerve repair. It was impressive that PUCL-alt-PEG group displayed stronger signals than the autograft group. This demonstrates that scaffolds of novel alternating block polyurethanes (PUCL-alt-PEG) show comparable or better nerve repair results than the autograft, which is considered as ‘gold standard’ in nerve repair.

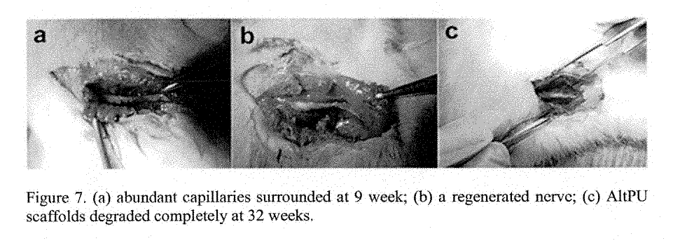

Histological Assessment: After the polyurethane scaffolds were dissected carefully under high magnification microsurgery at the 9th week postoperatively, a regenerated nerve was observed. The mature regenerated nerve tissues could be clearly observed as the PUCL-alt-PEG scaffolds degraded completely at 32 weeks post implantation (FIG. 7). No inflammatory signs or adverse tissue reactions were observed. The growth rate of the nerve matched very well with the degradation rate of the scaffold.

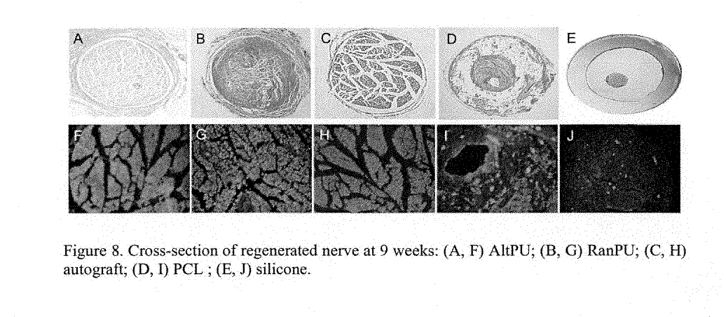

Immunofluorescent Staining: HE staining was employed to assess the morphology of regenerated nerves at the mid-section at the 9th week postoperatively (FIG. 8). It was observed that the neurofilaments grew rapidly along the entire space of PUCL-alt-PEG, PUCL-ran-PEG scaffolds and autografts. On the contrary, PCL scaffold and silicone tube showed less neurofilament growth. To observe axonal growth, Neurofilament-200 (NF-200) was used as a protein marker of axons.

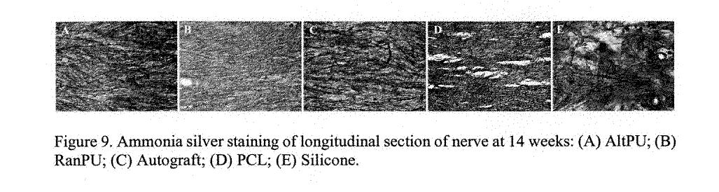

Ammonia silver staining, which was used to show regenerated nerve fibers and axons, demonstrated that axon myelin was nearly completely regenerated in the PUCL-alt-PEG, PUCL-ran-PEG scaffold and autograft groups with a bit of irregularity in their arrangements (FIG. 9). The axon myelin is almost completely regenerated and regularly spread throughout the PUCL-ran-PEG nerve guide scaffold compared with the autograft group at 14 weeks postoperatively. However, axon myelin showed little regeneration as well as a lack of regular arrangements in the PCL and silicone tube groups. The axon myelin also regenerated completely and spread regularly throughout the PUCL-alt-PEG scaffold group. In the autograft group, axon myelin generally regenerated well but showed a slight irregularity in their arrangement. Nerve regeneration in PUCL-alt-PEG scaffolds looked better than that of the autograft. The reasons may be in part due to the porous structures and high permeability of the PUCL-alt-PEG scaffolds, as the amphiphilic PU nerve guide scaffolds can readily allow nutrient and metabolites to permeate through the scaffold.

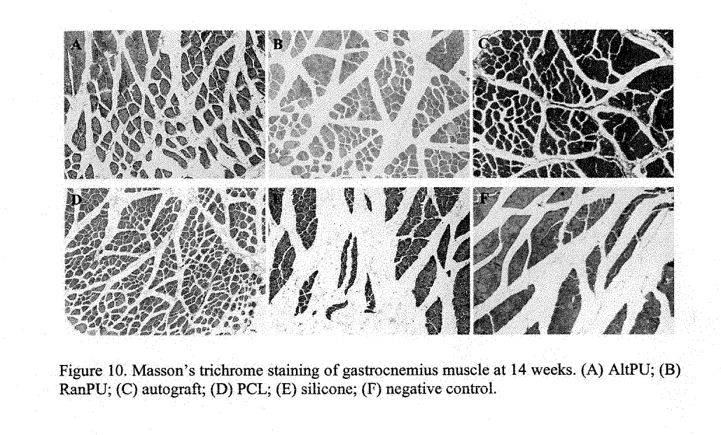

To evaluate the atrophy of rat gastrocnemius muscles resulting from dysfunction of the sciatic nerves, gastrocnemius muscles of rats in the six groups were stained with Masson's trichrome staining since gradual functional recovery of the sciatic nerves is accompanied by reduction of atrophy. Prominent reduction in muscle mass was obvious in rats with disrupted sciatic nerves that were implanted with silicone tubes, showing serious muscle atrophy (FIG. 10). In contrast, muscle atrophy was insignificant in rats implanted with PU nerve guidance conduits (NGC) and autograft. The average diameters of the muscle fibers in PU NGC and autograft were all larger than those of the fibers in PCL scaffold, silicone tube and negative control groups. PUCL-alt-PEG scaffold group had the highest average diameter of the muscle fiber, slightly larger than that of the autograft. From above HE staining, all results support the conclusion that PUCL-alt-PEG nerve guide scaffold provides the best nerve function repair capability among all the groups.

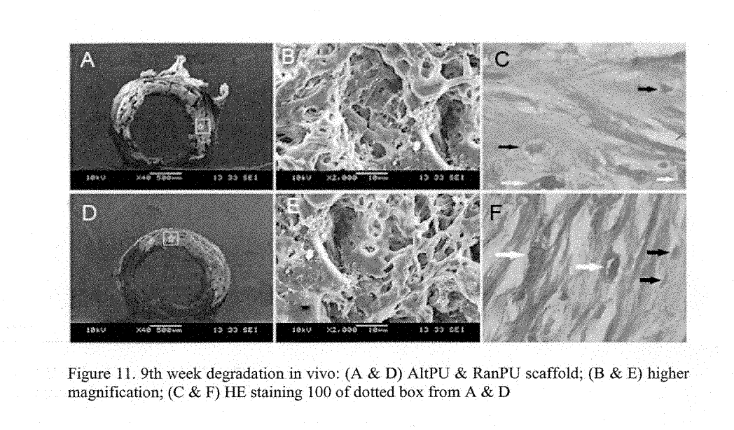

In vivo degradation of PUCL-alt-PEG and PUCL-ran-PEG nerve conduits after 9 weeks are shown in FIG. 11, which demonstrates significant degradation and tissue compatibility of the PUCL-alt-PEG nerve repair scaffolds, comparable to the PUCL-ran-PEG. According to the in vivo studies, degradation of the scaffolds at the 9th week was accompanied by invasion of blood vessels and connective tissue, indicating that the PU NGC can provide structural features adaptable to the physiological environment, and possess adequate strength and elasticity to allow regular motion of muscles around the conduit without resulting in scaffold collapse during degradation. Sciatic nerve cells, showing well-spread and flattened morphology, aligned themselves following the physical shape of the nerve guide scaffold, further demonstrating that PU NGC, being cytocompatible nerve conductive substrates, provide structural cues for the cells to take up the desired morphology. Blood vessels infiltrated into the PU NGC walls through the column-shaped micro-sized pores of the outer surface. The satisfactory nerve regeneration through PU scaffolds may be due to its porous structure and high permeability. Combined with its suitable mechanical properties, impressive nerve regeneration ability, and cytocompatibility, biodegradable PUCL-alt-PEG NGC show potential in clinical applications for peripheral nerve repair.

### Further Example Embodiments

The following are sample embodiments and are not intended to be limiting in any manner.

1. This is, for the first time to create a family of completely biodegradable block polyurethanes with alternating arrangement of the block segments. The macromolecular structure and architecture are new.

2. Based on Embodiment 1, the alternating block polyurethanes (AltPU) are a family of polymers including amphiphilic, hydrophilic and hydrophobic polymers with one hydrophilic block such as poly(ethylene glycol) (PEG), poly(propylene glycol) (PPG), their copolymers, and one or two or multiple aliphatic polyester blocks such as PCL, PLGA, PLA, PHA, PHB.

3. Based on Embodiment 2, the alternating block polyurethanes (AltPU) are made through a selectively coupling reaction between aliphatic polyester diols and diisocyanate-terminated hydrophilic polyether segments such as PEG, PPG, or between aliphatic polyether diols and diisocyanate-terminated aliphatic polyester diol, or between aliphatic polyester diols and diisocyanate-terminated aliphatic polyester segments such as PCL, PLGA, PLA, PHA, PHB.

4. Based on Embodiment 2, a PCL and PEG based alternating block polyurethanes (PUCL-alt-PEG)

5. Based on Embodiment 2, a PHA and PEG based alternating block polyurethanes (PUHB-alt-PEG)

6. Based on Embodiment 2, diisocyanate-terminated segments in the selectively coupling reaction are synthesized with all kinds of aliphatic diisocyanates including hexamethylenediisocyanate, lysine diisocyanate, triphenylmethane triisocyanate, isophoronediisocyanate, 4,4′-methylene bis(cyclohexyl isocyanate) etc.

7. Based on Embodiment 2, the selectively coupling reactions are catalyzed by tin catalysts such as tin(II) 2-ethylhexanoate (SnOct2), ditin butyldilaurate.

8. Based on Embodiment 2, the selectively coupling reactions are carried out either in hulk or in organic solvents under inert atmosphere.

9. The alternating block polyurethane design that is capable of creating a formation of more regular surfaced ravine-patterned structures, when compared to random block polyurethane designed polymers

10. The alternating block polyurethane design that is capable of creating an enhanced phase separation of the polymers, when compared to random block polyurethane designed polymers

11. The alternating block polyurethanes (AltPU) of Embodiment 2 with improved medical, mechanical and processing properties, minimum degradation pH change and well controlled degradation.

12. The materials of Embodiment 2, are used for nerve guidance conduits, and other soft and hard tissue regeneration and implantable medical devices.

13. Based on the materials of Embodiment 2, nerve repair conduits made from alternating block polyurethanes.

14. Based on the materials of Embodiment 2, a porous form of alternating block polyurethanes is prepared for use in nerve guidance conduits, and other soft and hard tissue regeneration and implantable medical devices based on the materials of Embodiment 2.

15. The products of Embodiment 14, such as the nerve guidance conduits, and other soft and hard tissue regeneration and implantable medical devices of alternating block polyurethanes (AltPU) are fabricated using methods such as salt leaching, freeze-drying, electrospinning, extrusion, molding, casting, and/or 3D printing.

### REFERENCES

[1] Santerre J P, Woodhouse K, Laroche G, Labow R S. Understanding the biodegradation of polyurethanes: From classical implants to tissue engineering materials. Biomaterials. 2005; 26:7457-70.

[2] Pan J, Li G, Chen Z, Chen X, Zhu W, Xu K. Alternative Block Polyurethanes Based on Poly(3-hydroxybutyrate-co-4-hydroxybutyrate) and Poly(ethylene glycol). Biomaterials. 2009; 30:2974-84.

[3] Li G, Liu, Y, Li, D, Zhang, L, Xu K. A Comparative Study on Structure-Property Elucidation of P3/4HB and PEG Based Block Polyurethanes. Journal of Biomedical Materials Research: Part A. 2012; 100:2319-29.

[4] Li G, Li D, Niu Y, He T, Chen K, Xu K. Alternating Block Polyurethanes Based on PCL and PEG as Potential Nerve Regeneration Materials. Journal of Biomedical Materials Research: Part A. 2014; 102:685-97.

[5] Li G, Liu P, Qiu H, Li D, Xu K. Synthesis, Characterizations and Biocompatibility of Alternating Block Polyurethanes Based on P3/4HB and PPG-PEG-PPG. Journal of Biomedical Materials Research: Part A. 2011; 98:88-9.

[6] Niu Y, Chen K, He T, Yu W, Huang S, Xu K. Scaffolds from Block Polyurethanes Based on Poly(ε-caprolactone) (PCL) and Poly(ethylene glycol) (PEG) for Peripheral Nerve Regeneration. Biomaterials. 2014; 35:4266-77.

[7] Niu Y, Li L, Chen K, Chen F, Liu, X, Ye, J, Li W, Xu K. Scaffolds from Alternating Block Polyurethanes of PCL and PEG with Stimulation and Guidance of Nerve Growth and Better Nerve Repair than Autograft. Journal of Biomedical Materials Research: Part A. 2015; 103:2355-64.

[8] DenDunnen W F A, VanderLei B, Schakenraad J M, Stokroos I, Blaauw E, Bartels H, et al. Poly(DL-lactide-epsilon-caprolactone) nerve guides perform better than autologous nerve grafts. Microsurgery. 1996; 17:348-57.

[9] Luis A L, Rodrigues J M, Lobato J V, Lopes M A, Amado S, Veloso A P, et al. Evaluation of two biodegradable nerve guides for the reconstruction of the rat sciatic nerve. Biomed Mater Eng. 2007; 17:39-52.

[10] Johnson E O, Soucacos P N. Nerve repair: experimental and clinical evaluation of biodegradable artificial nerve guides. Injury. 2008; 39 Suppl 3:S30-6.

[11] Hoppen H J, Leenslag J W, Pennings A J, van der Lei B, Robinson P H. Two-ply biodegradable nerve guide: basic aspects of design, construction and biological performance. Biomaterials. 1990; 11:286-90.

[12] Meek M F, Varejao A S, Geuna S. Muscle grafts and alternatives for nerve repair. J Oral Maxillofac Surg. 2002; 60:1095-6; author reply 6.

[13] Johnson E O, Soucacos P N. Nerve repair: experimental and clinical evaluation of biodegradable artificial nerve guides. Injury. 2008; 39 Suppl 3:S30-6.

[14] Angiusa D, Wanga H, Spinnera R J, Gutierrez-Cottob Y, Yaszemskic M J, Windebankd A J. A systematic review of animal models used to study nerve regeneration in tissue-engineered scaffolds. Biomaterials. 2012; 32:8034=9.

[15] Wang X, Cui T, Yan Y, Zhang R. Peroneal Nerve Regeneration Using a Unique Bilayer Polyurethane-collagen Guide Conduit. Journal of Bioactive and Compatible Polymers 2009; 24:109-27.

[16] Yin D, Wang X H, Yan Y, Zhang R. Preliminary Studies on Peripheral Nerve Regeneration using a New Polyurethane Conduit. Journal of Bioactive and Compatible Polymers. 2007; 22:143-59.

[17] Tran R T, Choy W M, Cao H, Qattan I, Chiao J C, Ip W Y, et al. Fabrication and characterization of biomimetic multichanneled crosslinked-urethane-doped polyester tissue engineered nerve guides. J Biomed Mater Res A. 2013.

[18] G Y Li, D D Li, Y Q Niu, T He, K C Chen, Xu K. Alternating block polyurethanes based on PCL and PEG as potential nerve regeneration materials. J Biomed Mater Res A. 2014; 102:685-97.

1-6. (canceled)

7. A medical device formed from the block copolymer with alternating arrangement of the block segments, but also including random arrangement of the block segments. The biodegradable block polyurethanes comprise first blocks and second blocks; and wherein first blocks and second blocks are linked via urethane bonds. The first blocks comprise a diol-terminated aliphatic polyester, and the second blocks comprise a two diisocyanate-terminated hydrophilic polymer or oligomer

8. A medical device of claim 1, wherein the medical device is a peripheral nerve guidance conduit. The peripheral nerve guidance is formed from the biodegradable alternating block polyurethanes. The alternating structure is created via selectively coupling reaction between a diol-terminated aliphatic polyester and aliphatic diisocyanate-terminated polymer or oligomer. The diol-terminated aliphatic polyesters include polycaprolactone (PCL), poly(D,L-lactic-co-glycolic acid) (PLGA), poly(lactic acid) (PLA), polyhydroxyalkanoate (PHA), poly(lactic acid)-polyethylene glycol) copolymer (PLAPEG), polyhydroxybutyrate (PHB), or a combination thereof. The diisocyanate-terminated polymer or oligomer comprise a two diisocyanate-terminated polyethylene glycol (PEG), polypropylene glycol (PPG), polytertahydrofuran (PTHF), or a combination thereof.

9. The medical device of claim 2, wherein the nerve guidance conduit has a porous hollow structure with porosity degree of 10-99% and pore sizes of 100 nm to 500μ (micrometer).

10. The medical device of claim 2, wherein the nerve guidance conduit contains bioactive substances such as protein RGD, nerve growth factor (NGF), nerve growth drug, Swann cell and other nerve beneficial substance.
